(19) United States
(12) Patent Application Publication
(10) Pub. No.: US 2016/0129343 A1
(43) Pub. Date: May 12, 2016

(54) REHABILITATIVE POSTURE AND GESTURE RECOGNITION

(71) Applicant: BIOGAMING LTD., Petah-Tikva (IL)

(72) Inventors: Arkady DOMANSKY, Ramat Gan (IL); Ido AZRAN, Tel Aviv (IL); Eytan MAJAR, Carmei Yosef (IL)

(21) Appl. No.: 14/897,251

(22) PCT Filed: Jun. 12, 2014
(86) PCT No.: PCT/IL2014/050536

§ 371 Date: Dec. 10, 2015

(30) Foreign Application Priority Data
Jun. 13, 2013 (GB) 1310515.0

(51) Int. Cl. A63F 13/21; A61B 5/00; A61B 5/11; G06K 9/00; H04N 5/77

(52) U.S. Cl. CPC A63F 13/21; G06K 9/00342; H04N 5/77; G06K 9/00375; A61B 5/11; A61B 5/486

(57) ABSTRACT

A kinetic rehabilitation system comprising: a kinetic sensor comprising a motion-sensing camera; and a computing device comprising: (a) a non-transient memory comprising a stored set of values of rehabilitative gestures each defined by a time series of spatial relations between a plurality of theoretical body joints, and wherein each time series comprises: initial spatial relations, mid-gesture spatial relations and final spatial relations, and (b) a hardware processor configured to continuously receive a recorded time series of frames from said motion-sensing camera, wherein each frame comprises a three-dimensional position of each of a plurality of body joints of a patient, wherein said hardware processor is further configured to compare, in real time, at least a portion of the recorded time series of frames with the time series of spatial relations, to detect a rehabilitative gesture performed by the patient.

### FIELD OF THE INVENTION

The invention relates to rehabilitative posture and gesture recognition.

### BACKGROUND

Decline in physical function is often associated with age-related impairments to overall health, or may be the result of injury or disease. Such a decline contributes to parallel declines in self-confidence, social interactions and community involvement. People with motor disabilities often experience limitations in fine motor control, strength, and range of motion. These deficits can dramatically limit their ability to perform daily tasks, such as dressing, hair combing, and bathing, independently. In addition, these deficits, as well as pain, can reduce participation in community and leisure activities, and even negatively impact occupation.

Participating in and complying with physical therapy, which usually includes repetitive exercises, is an essential part of the rehabilitation process which is aimed to help people with motor disabilities overcome the limitations they experience. However, it has been argued that most of the people with motor disabilities do not perform the exercises as recommended. People often cite a lack of motivation as an impediment to them performing the exercises regularly. Furthermore, the number of exercises in a therapy session is oftentimes insufficient. During rehabilitation, the therapist usually personally provides physical assistance and monitors whether each student's movements are reaching a specific standard. Thus, the therapist can only rehabilitate one patient at a time, or a small group of patients at most. Patients often lack enthusiasm to participate in the tedious rehabilitation process, resulting in continued muscle atrophy and insufficient muscle endurance.

Also, it is well known that adults and especially children get bored repeating the same movements. This can be problematic when an adult or a child has to exercise certain muscles during a post-trauma rehabilitation period. For example, special exercises are typically required after a person breaks his or her arm. It is hard to make this repetitive work interesting. Existing methods to help people during rehabilitation include games to encourage people, and especially children, to exercise more.

Therefore, it is highly advantageous for patients to perform rehabilitative physical therapy at home, using techniques to make repetitive physical exercises more entertaining. Uses of video games technologies are beginning to be explored as a commercially available means for delivering training and rehabilitation programs to patients in their own homes.

U.S. Pat. No. 6,712,692 to Basson et al. discloses a method for gathering information about movements of a person, which could be an adult or child. This information is mapped to one or more game controller commands. The game controller commands are coupled to a video game, and the videogame responds to the game controller commands as it would normally.

U.S. Pat. No. 7,996,793 to Latta et al. discloses Systems, methods and computer readable media for gesture recognizer system architecture. A recognizer engine is provided, which receives user motion data and provides that data to a plurality of filters. A filter corresponds to a gesture, which may then be tuned by application receiving information from the gesture recognizer so that the specific parameters of the gesture-such as arm acceleration for a throwing gesture may be set on a per-application level, or multiple times within a single application. Each filter may output to an application using it a confidence level that the corresponding gesture occurred, as well as further details about the user motion data.

U.S. Patent Application No. 2012/0190505A1 to Shavit et al. discloses a system for monitoring performance of a physical exercise routine comprises a Pilates exercise device enabling a user to perform the physical exercise routine, a plurality of motion and position sensors for generating sensory information that includes at least position and movements of a user performing the physical exercise routine; a database containing routine information representing at least an optimal execution of the physical exercise routine; a training module configured to separate from sensory information at least appearance of the Pilates exercise device, compare the separated sensory information to the routine information to detect at least dissimilarities between the sensory information and the routine information, wherein the dissimilarities indicate an incorrect execution of the physical exercise routine, the training module is further configured to feedback the user with instructions related to correcting the execution of the physical exercise routine by the user; and a display for displaying the feedback.

Smith et al. (2012) disclose an overview of the main videogame console systems (Nintendo Wii™, Sony Playstation® and Microsoft Xbox®) and discussion of some scenarios where they have been used for rehabilitation, assessment and training of functional ability in older adults. In particular, two issues that significantly impact functional independence in older adults are injury and disability resulting from stroke and falls. See S. T. Smith, D. Schoene, The use of Exercise-based Videogames for Training and Rehabilitation of Physical Function in Older Adults, Aging Health. 2012;8(3):243-252.

Ganesan et al. (2012) disclose a project that aims to find the factors that play an important role in motivating older adults to maintain a physical exercise routine, a habit recommended by doctors but difficult to sustain. The initial data gathering includes an interview with an expert in aging and physical therapy, and a focus group with older adults on the topics of exercise and technology. Based on these data, an early prototype game has been implemented for the Microsoft Kinect that aims to help encourage older adults to exercise. The Kinect application has been tested for basic usability and found to be promising. Next steps include play-tests with older adults, iterative development of the game to add motivational features, and evaluation of the game's success in encouraging older adults to maintain an exercise regimen. See S. Ganesan, L. Anthony, Using the Kinect to encourage older adults to exercise: a prototype, in Extended Abstracts of the ACM Conference on Human Factors in Computing Systems (CHI'2012), Austin, Tex., 5 May 2012, p.2297-2302.

Lange et al. (2011) disclose that the use of the commercial video games as rehabilitation tools, such as the Nintendo WiiFit, has recently gained much interest in the physical therapy arena. Motion tracking controllers such as the Nintendo Wiimote are not sensitive enough to accurately measure performance in all components of balance. Additionally, users can figure out how to “cheat” inaccurate trackers by performing minimal movement (e.g. wrist twisting a Wiimote instead of a full arm swing). Physical rehabilitation requires accurate and appropriate tracking and feedback of performance. To this end, applications that leverage recent advances in commercial video game technology to provide full-body control of animated virtual characters are developed. A key component of the approach is the use of newly available low cost depth sensing camera technology that provides markerless full-body tracking on a conventional PC. The aim of the research was to develop and assess an interactive game-based rehabilitation tool for balance training of adults with neurological injury. See B. Lange, C. Y. Chang, E. Suma, B. Newman, A. S. Rizzo, M. Bolas, Development and evaluation of low cost game-based balance rehabilitation tool using the Microsoft Kinect sensor, 33rd Annual International Conference of the IEEE EMBS, 2011.

Differently from “regular” garners, for patients who use video games for physiotherapy and rehabilitation purposes there is a great significance to the accuracy of postures and gestures, and for the correct way of performing the exercises.

Shen (2012) discloses a natural user interface to control the visualizer—“Visual Molecule Dynamics” using the Microsoft Kinect. The related background of human-computer interaction, image processing, pattern recognition and computer vision are introduced. An original algorithm was desinged for counting the finger number of the hand shape, which depends on the binarilization of depth image and the morphology binary processing. A Bayesian classifier was designed and implemented for the gesture recognition tasks. See Chen Shen, Controlling Visual Molecule Dynamics using Microsoft Kinect, the University of Edinburgh, 2012.

Lopez (2012) discusses the problem of Human Gesture Recognition using Human Behavior Analysis technologies. In particular, he applies the proposed methodologies in both health care and social applications. In these contexts, gestures are usually performed in a natural way, producing a high variability between the Human Poses that belong to them. This fact makes Human Gesture Recognition a very challenging task, as well as their generalization on developing technologies for Human Behavior Analysis. In order to tackle with the complete framework for Human Gesture Recognition, he split the process in three main goals: Computing multi-modal feature spaces, probabilistic modelling of gestures, and clustering of Human Poses for Sub-Gesture representation. Each of these goals implicitly includes different challenging problems, which are interconnected and faced by three presented approaches: Bag-of-Visual-and-Depth-Words, Probabilistic-Based Dynamic Time Warping, and Sub-Gesture Representation. The methodologies of each of these approaches are explained in detail. He has validated the presented approaches on different public and designed data sets, showing high performance and the viability of using our methods for real Human Behavior Analysis systems and applications. Finally, he shows a summary of different related applications currently in development, as well as both conclusions and future trends of research. See Victor Ponce Lopez, Multi-Modal Human Gesture Recognition Combining Dynamic Programming and Probabilistic Methods, Master of Science Thesis, Barcelona, 2012.

The foregoing examples of the related art and limitations related therewith are intended to be illustrative and not exclusive. Other limitations of the related art will become apparent to those of skill in the art upon a reading of the specification and a study of the figures.

### SUMMARY

The following embodiments and aspects thereof are described and illustrated in conjunction with systems, tools and methods which are meant to be exemplary and illustrative, not limiting in scope.

There is provided, in accordance with an embodiment, a kinetic rehabilitation system comprising: a kinetic sensor comprising a motion-sensing camera; and a computing device comprising: (a) a non-transient memory comprising a stored set of values of rehabilitative gestures each defined by a time series of spatial relations between a plurality of theoretical body joints, and wherein each time series comprises: initial spatial relations, mid-gesture spatial relations and final spatial relations, and (b) a hardware processor configured to continuously receive a recorded time series of frames from said motion-sensing camera, wherein each frame comprises a three-dimensional position of each of a plurality of body joints of a patient, wherein said hardware processor is further configured to compare, in real time, at least a portion of the recorded time series of frames with the time series of spatial relations, to detect a rehabilitative gesture performed by the patient.

There is further provided, in accordance with an embodiment, a method for gesture detection in a kinetic rehabilitation system, the method comprising: providing a stored set of values of rehabilitative gestures each defined by time series of spatial relations between a plurality of theoretical body joints, wherein each time series comprises: initial spatial relations, mid-gesture spatial relations and final spatial relations; and using at least one hardware processor for:(a) continuously receiving a recorded time series of frames from a motion-sensing camera, wherein each frame comprises a three-dimensional position of each of a plurality of body joints of a patient, and (b) comparing, in real time, at least a portion of the recorded time series of frames with the time series of spatial relations, to detect a rehabilitative gesture performed by the patient.

In some embodiments, each of said time series of spatial relations further comprises one or more range values for each of at least one of said spatial relations.

In some embodiments, said time series of spatial relations further comprises one or more range values for the transition time between each of at least one of said spatial relations.

In some embodiments, said spatial relations each comprise angles between vectors formed in a three-dimensional space by said theoretical body joints.

In some embodiments, said spatial relations comprise distances in a three-dimensional space between said theoretical body joints.

In some embodiments, said motion-sensing camera is configured to yield said recorded time series of frames at a frame rate of 20 frames per second or more.

In some embodiments, said motion-sensing camera is configured to yield said recorded time series of frames at a frame rate of 30 frames per second or more.

In some embodiments, said motion-sensing camera is configured to yield said recorded time series of frames at a frame rate of 40 frames per second or more.

In some embodiments, said hardware processor is further configured to convert said three-dimensional position in said recorded time series of frames to angles between vectors formed in a three-dimensional space by said theoretical body joints.

In some embodiments, said hardware processor is further configured to convert said three-dimensional position in said recorded time series of frames to distances in a. three-dimensional space between said theoretical body joints.

In addition to the exemplary aspects and embodiments described above, further aspects and embodiments will become apparent by reference to the figures and by study of the following detailed description.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. The figures are listed below.

FIG. 1 shows a block diagram of the system for rehabilitative treatment, in accordance with some embodiments;

FIG. 2 shows an example of a dedicated web page which summarizes information on a certain patient, in accordance with some embodiments;

FIG. 3 shows an example of a dedicated web page which is utilized by the therapist to construct a therapy plan for a certain patient, in accordance with some embodiments;

FIG. 4 shows an illustration of a structured light method for depth recognition, in accordance with some embodiments;

FIG. 5 shows a top view 2D illustration of a triangulation calculation used for determining a pixel depth, in accordance with some embodiments;

FIG. 6 shows an illustration of a human primary body parts and joints, in accordance with some embodiments;

FIG. 7 shows an example of one video game level screen shot, in accordance with some embodiments;

FIG. 8 shows an example of another video game level screen shot, in accordance with some embodiments;

FIG. 9 shows an illustration of a right lunge exercise monitoring, in accordance with some embodiments;

FIG. 10 shows an illustration of a right pendulum exercise monitoring, in accordance with some embodiments;

FIG. 11 shows an illustration of a double leg jump exercise monitoring, in accordance with some embodiments;

FIG. 12 shows an illustration of a left leg jump monitoring, in accordance with some embodiments; and

FIG. 13 shows a block diagram of a gesture detection method, in accordance with some embodiments.

### DETAILED DESCRIPTION

Disclosed herein are a system and a method for gesture detection in a kinetic rehabilitation system.

Conventionally, people who require rehabilitative therapy, such as accident victims who suffered physical damages and need physiotherapeutic treatment, elderly people who suffer from degenerative diseases, children who suffer from physically-limiting cerebral palsy, etc., arrive to a rehabilitation center, meet with a therapist who prescribes a therapy plan for them, and execute the plan at the rehabilitation center and/or at home. In many cases, the therapy plan comprises of repeatedly-performed physical exercises, with or without therapist supervision. The plan normally extends over multiple appointments, when in each appointment the therapist may monitor the patient's progress and raise the difficulty level of the exercises. This conventional method has a few drawbacks: it requires the patient's arrival to the rehabilitation center, at least for a portion of the plan, which may be time consuming and difficult for some people (e.g. elderly people, small children, etc,), it often involves repetitive and boring activity, which may lead to lack of motivation and abandonment of the plan, and may limit the therapist to treat a rather small number of patients.

Thus, allowing the executing a therapy plan in the form of a video game, at the convenience of the patient's home, with easy communication between therapists and patients for plan prescribing and progress monitoring, may be highly advantageous to both therapists and patients. Moreover, combining the aforementioned advantages while providing for patient-specific video games, rather than generic video games, is also of great significance.

Nevertheless, for achieving efficient therapy using video games, the exercises need to be performed with care to movement accuracy, performance duration, etc. Currently, many regular interactive video games which utilize a motion recognition device do not take such parameters into consideration, mostly because such accuracy is not needed for regular video games. Hence, a system and method for accurate recognizing of postures and gestures may be also advantageous.

### GLOSSARY

Video game: a game for playing by a human player, where the main interface to the player is visual content displayed using a monitor, for example. A video game may be executed by a computing device such as a personal computer (PC) or a dedicated gaming console, which may be connected to an output display such as a television screen, and to an input controller such as a handheld controller, a motion recognition device, etc.

Level of video game: a confined part of a video game, with a defined beginning and end. Usually, a video game includes multiple levels, where each level may involve a higher difficulty level and require more effort from the player.

Video game controller: a hardware part of a user interface (UI) used by the player to interact with the PC or gaming console.

Kinetic sensor: a type of a video game controller which allows the user to interact with the PC or gaming console by way of recognizing the user's body motion. Examples include handheld sensors which are physically moved by the user, body-attachable sensors, cameras which detect the user's motion, etc.

Motion recognition device: a type of a kinetic sensor, being an electronic apparatus used for remote sensing of a player's motions, and translating them to signals that can be input to the game console and used by the video game to react to the player motion and form interactive gaming.

Motion recognition game system: a system including a PC or game console and a motion recognition device.

Video game interaction: the way the user instructs the video game what he or she wishes to do in the game. The interaction can be, for example, mouse interaction, controller interaction, touch interaction, close range camera interaction or long range camera interaction.

Gesture: a physical movement of one or more body parts of a player, which may be recognized by the motion recognition device.

Exercise: a physical activity of a specific type, done for a certain rehabilitative purpose. An exercise may be comprised of one or more gestures. For example, the exercise referred to as “lunge”, in which one leg is moved forward abruptly, may be used to strengthen the quadriceps muscle, and the exercise referred to as “leg stance” is may be used to improve stability, etc.

Repetition (also “instance”): one performance of a certain exercise. For example, one repetition of a leg stance exercise includes gestures which begin with lifting one leg in the air, maintaining the leg in the air for a specified period of time, and placing the leg back on the ground.

Intermission: A period of time between two consecutive repetitions of an exercise, during which period the player may rest.

One example for a suitable motion recognition device is the Microsft Corp. Kinect, a motion-sensing camera for the Xbox 360 video game console and Windows PCs. Based around a webcam-style add-on peripheral for the Xbox 360 console, the Kincet enables users to control and interact with the Xbox 360 using a kinetic UI, without the need to touch a game controller, through a natural user interface using physical gestures.

The present system and method may also be adapted to other gaming consoles, such as Sony PlayStation, Nintendo Wii, etc., and the motion recognition device may be a standard device for these or other gaming consoles.

Unless specifically stated otherwise, as apparent from the following discussions, it is appreciated that throughout the specification discussions utilizing terms such as “processing”, “computing”, “calculating”, “determining”, or the like, refer to the action and/or process of a computing system or a similar electronic computing device, that manipulate and/or transform data represented as physical, such as electronic, quantities within the computing system's registers and/or memories into other data similarly represented as physical quantities within the computing system's memories, registers or other such.

Some embodiments may be implemented, for example, using a computer-readable medium or article which may store an instruction or a set of instructions that, if executed by a computer (for example, by a hardware processor and/or by other suitable machines), cause the computer to perform a method and/or operations in accordance with embodiments of the invention. Such a computer may include, for example, any suitable processing platform, computing platform, computing device, processing device, computing system, processing system, computer, processor, gaming console or the like, and may be implemented using any suitable combination of hardware and/or software. The computer-readable medium or article may include, for example, any type of disk including floppy disks, optical disks, CD-ROMs, magnetic-optical disks, read-only memories (ROMs), random access memories (RAMs), flash memories, electrically programmable read-only memories (EPROMs), electrically erasable and programmable read only memories (EEPROMs), magnetic or optical cards, or any other type of media suitable for storing electronic instructions, and capable of being coupled to a computer system bus.

The instructions may include any suitable type of code, for example, source code, compiled code, interpreted code, executable code, static code, dynamic code, or the like, and may be implemented using, any suitable high-level, low-level, object-oriented, visual, compiled and/or interpreted programming language, such as C, C++, C#, Java, BASIC, Pascal, Fortran, Cobol, assembly language, machine code, or the like.

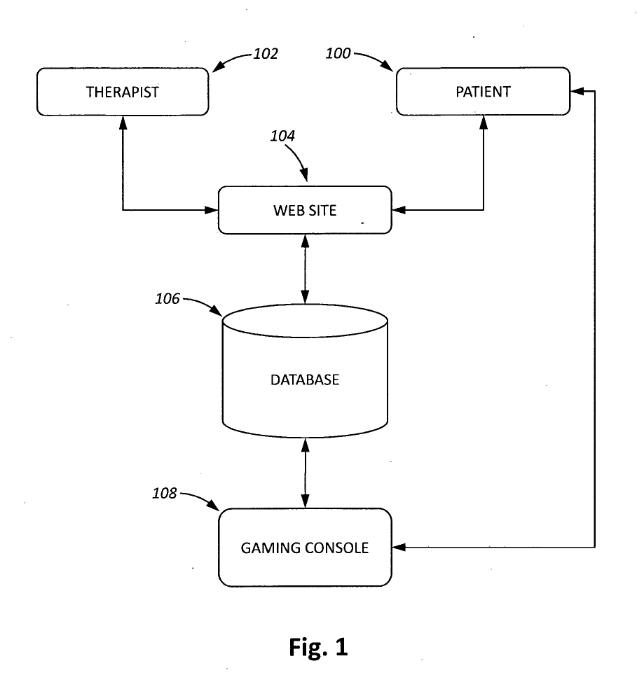

The present system and method may be better understood with reference to the accompanying figures. Reference is now made to FIG. 1, which shows a block diagram of the system for rehabilitative treatment. The therapist 102 may logon to the dedicated web site 104, communicate with patients 100, prescribe therapy plans (also referred to as “prescriptions” or “treatment plans”), and monitor patient progress. Web site 104 may receive the prescribed plan and store it in a dedicated database 106. The therapy plan than may be automatically translated to a video game level. When patient 100 activates his or her video game, the new level, or instructions for generating the new level, may be downloaded to his or her gaming console 108 and he or she may play this new level. Since the game may be interactive, the motion recognition device may monitor the patient movements for storing patient results and progress, and or for providing real time feedback during the game play, such as in the form of score accumulation. The results, in turn, may be sent to database 106 for storage and may be available for viewing on web site 104 by therapist 102 for monitoring patient 100 progress, and to patient 100 for receiving feedback.

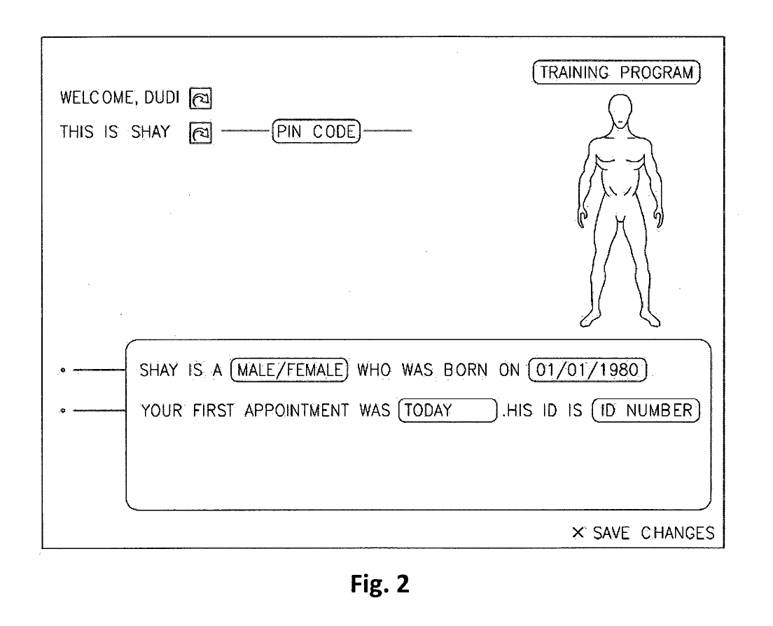

Reference is now made to FIG. 2, which shows an example of a dedicated web site page which summarizes information on a certain patient for the therapist. The page may display a summary of the patient profile, appointments history, diagnosis, other therapists comment history, etc.

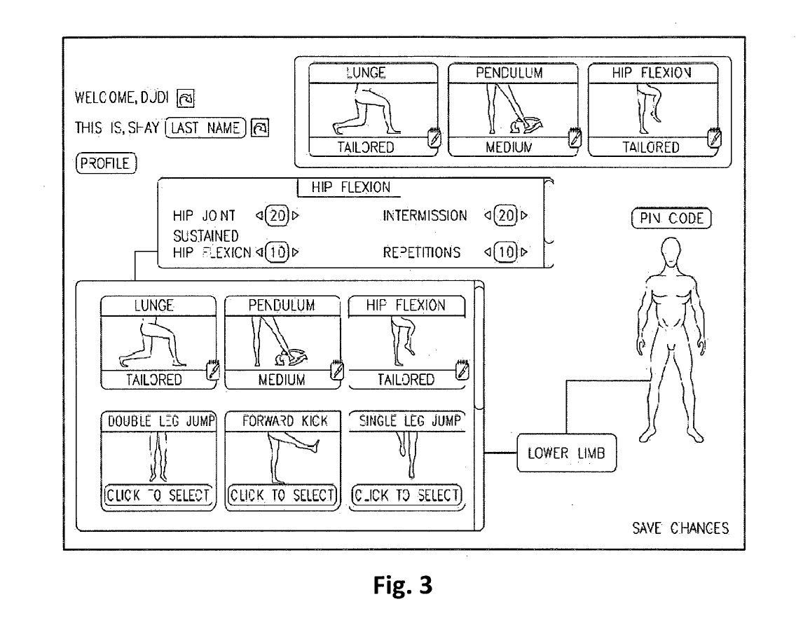

Reference is now made to FIG. 3, which shows an example of a dedicated web site page which is utilized by the therapist to construct a therapy plan for a certain patient. The therapist may input the required exercises, repetition number, difficulty level, etc. Since the use of motion recognition device may be significant for the present method, the principle of operation of a commercially-available motion recognition device (Kinect) and its contribution to the method is described hereinafter.

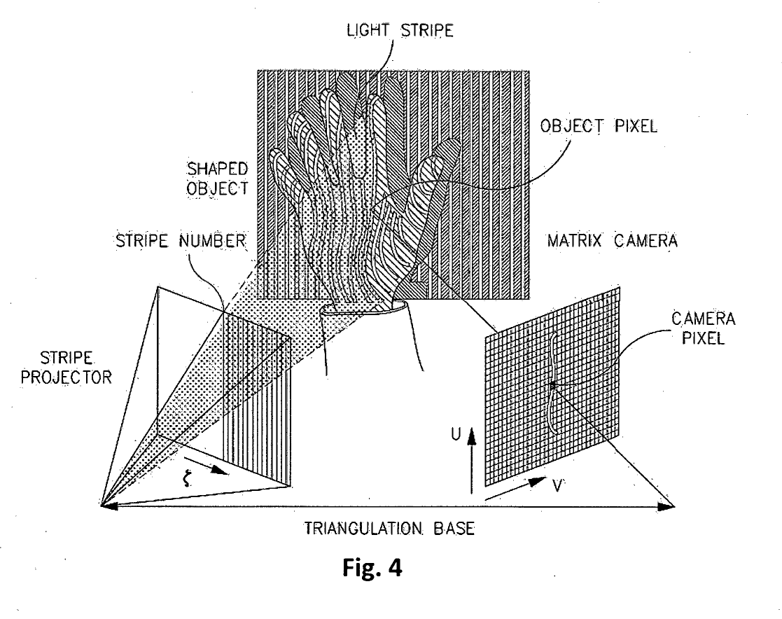

Reference is now made to FIG. 4, which shows an illustration of a structured light method for depth recognition. A projector may be used for projecting the scene with known stripe-like light pattern. The projected object may distort the light pattern with equivalency to its shape. A camera, which may be installed at a known distance from the projector, may then capture the light reflected from the object and sense the distortion that may be formed in the light pattern, and the angle of the reflected light, for each pixel . of the image.

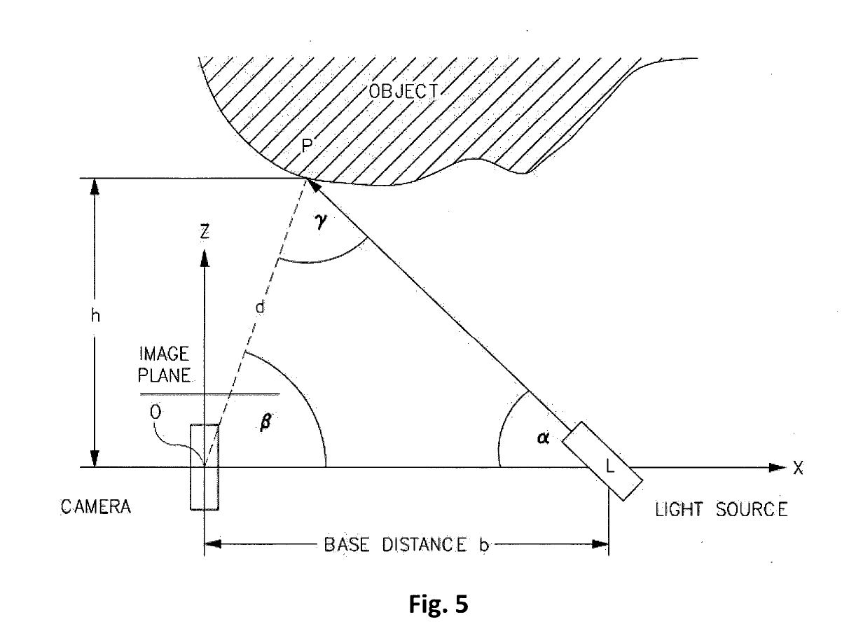

Reference is now made to FIG. 5, which shows a top view 2D illustration of a triangulation calculation used for determining a pixel depth. The camera may be located in a known distance from the light source (b). P is a point on the projected object which coordinates are to be calculated. According to the law of sines:

$$\frac{d}{sin α} = \frac{b}{sin γ} {}^{yields} d = \frac{b \cdot sin α}{sin γ} = \frac{b \cdot α}{sin π - α - β} = \frac{b \cdot α}{sin α + β} ,$$

and P coordinates are given by (d cos β, d sin β). Since α and b are known, and β is defined by the projective geometry, P coordinates may be resolved. The above calculation is made for 2D for the sake of simplicity, but the real device may actually calculate a 3D solution for each pixel coordinates to form a complete depth image of the scene, which may be utilized to recognize human movements.

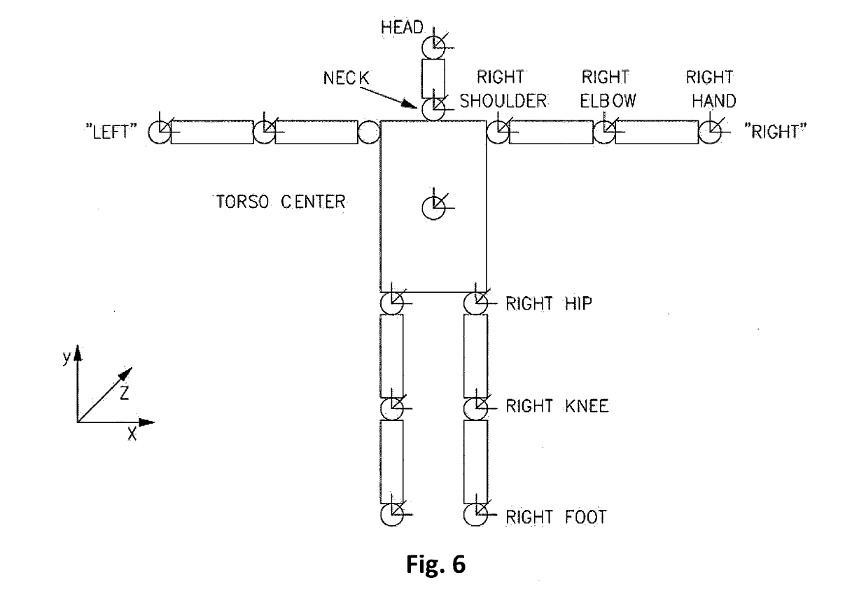

Reference is now made to FIG. 6, which shows an illustration of human primary body parts and joints. By recognizing the patient body parts and joints movements, the discussed method may enable to analyze the patient gestures and responses to the actions required by the game, for yielding an immediate feedback for the patient, and for storage for future analysis by the therapist.

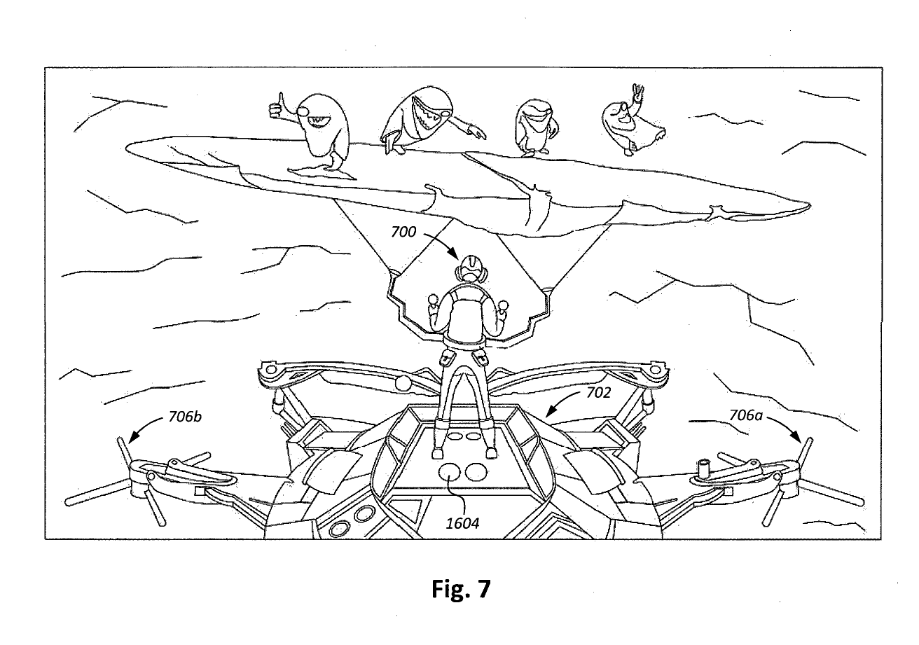

Reference is now made to FIG. 7, which shows one example of a video game level screen shot. This specific level may be designed to include squats, lunges, kicks, leg pendulums, etc. The patient may see a character 700 performing his own movements at real time. Character 700 may stand on a moving vehicle 702, which may accelerate when the patient is performing squats, and may slow when the patient lunges. Some foot spots 704 may be depicted on vehicle 702 platform and may be dynamically highlighted, in order to guide the patient to place his feet in the correct positions while performing the squats, lunges, kicks, etc. Right rotating device 706a and left rotating device 706b may be depicted on the right and left sides of vehicle 702, to form a visual feedback for the patient while performing leg pendulum exercises.

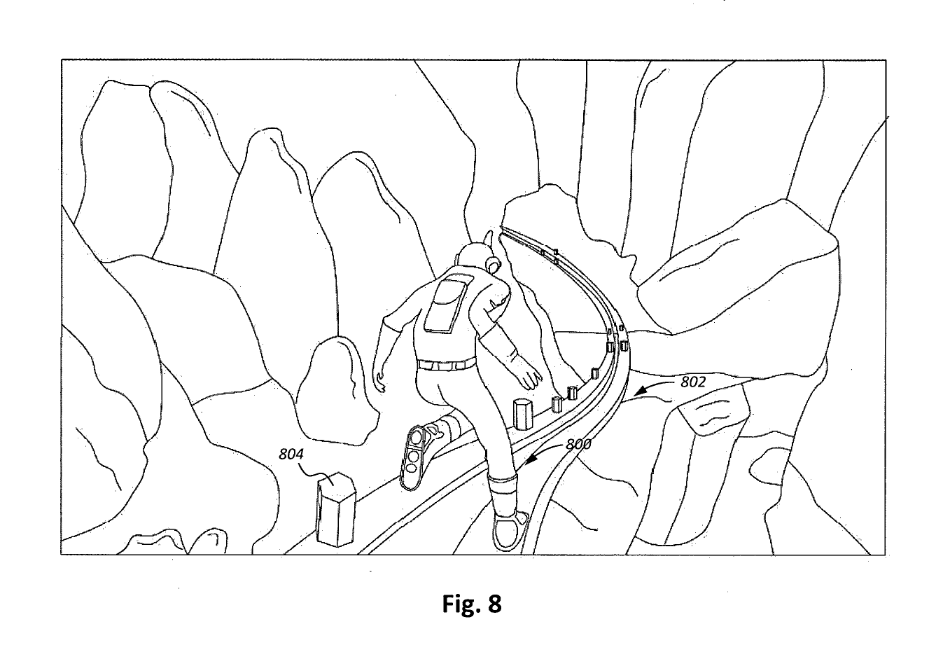

Reference is now made to FIG. 8, which shows another example of a video game level screen shot. This specific level may be designed to include hip flexions, leg stances and jumps, etc. The patient may see a character 800 performing his own movements at real time. Character 800 may advance on a rail 802 planted with obstacles 804. The patient may need to perform actions such as hip flexion, leg jump, etc., to avoid the obstacles and/or collect objects.

### Joints Mutual Relation Calculation

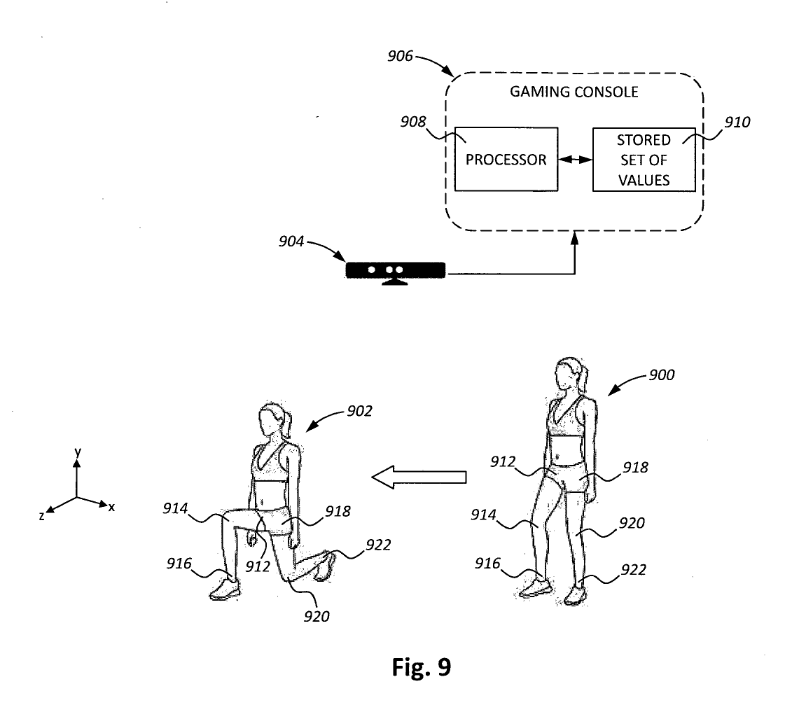

Reference is now made to FIG. 9, which shows an illustration of a right lunge exercise monitoring. A patient in a lunge initial posture 900 may perform a lunge exercise, which may end in a lunge final posture 902. Patient movement may be monitored by a motion recognition device (e.g. Kinect) 904 by way of sampling location of a plurality of body joints in a three dimensional space (i.e. x,y,z coordinates), within each frame it captures. A series of frames may then be transferred at a frame rate which may be 20, 30, 40 frames per second or more to a computing device such as a gaming console 906.

Gaming console 906 may include a processor 908 and a stored set of values 910 in order to compute and translate patient movement to distinguished postures and gestures. Processor 908 may convert locations of body joints in a three dimensional space (i.e. x,y,z coordinates) to spatial relations between body limbs and/or joints (i.e. distances between limbs and/or joints, and/or angles between vectors temporally formed by limbs and/or joints) for each captured frame. The calculation results may then be compared to stored set of values 910. These values may define the required spatial relations between body limbs and/or joints (i.e. the required range for distances between limbs and/or joints, and/or angles between vectors formed by limbs and/or joints) for an appropriate performing of a specific exercise at any phase of its execution (including start and end of exercise).

In addition, stored set of values 910 may also store range values for the transition time between spatial relations required to appropriately perform the exercise within its different phases. In the depicted example, for appropriate performance of a lunge, a certain initial posture 900 may be required. Processor 908 may calculate spatial distances and/or angles between right hip joint 912, right knee 914 and right ankle 916 in the following way: a vector between right hip joint 912 and right knee 914 may be calculated, by subtracting their spatial positions. Similarly, a vector between right knee 914 and right ankle 916 may be calculated. Finally, a spatial angle between these vectors may be calculated, to verify that these joints may be approximately aligned on one line (i.e. patient right leg is approximately straight). Similarly, left hip joint 918, left knee 920 and left ankle 922 may be also required to be approximately aligned on one line (i.e. patient left leg is straight). Also, right knee 914 and left knee 920 may be required to be aligned (i.e. none of them should stick out forward), within a certain distance between them.

A certain final posture 902 may be required as well. Processor 908 may calculate spatial distances and/or angles between right hip joint 912 and right knee 914 in the following way: a vector between right hip joint 912 and right knee 914 may be calculated, by subtracting their spatial positions. This vector may be required to be parallel to the floor, which is, for example, an XZ plane whose Y value equals zero. Similarly, a vector between right knee 914 and right ankle 916 may be calculated. This vector may be required to be perpendicular to the floor. Finally, a spatial angle between these vectors may be calculated, to verify that they may form a 90°±10° angle between them (i.e. patient right shin is 90°±10° bent in relation to the right hip). Similarly, the vector between left hip joint 918 and left knee 920, may be required to be perpendicular to the floor. Finally, right knee 914 and left knee 920 may be required to be within a certain distance (i.e. patient knees are not inbound or outbound). It should be noticed that when in final posture 902, left ankle 922 might be concealed from motion recognition device 904 by left knee 920 and/or left hip. In this situation, motion recognition device 904 may mistakenly transfer false left ankle 922 position (e.g. under the floor level), or transfer no position at all. The system may detect this situation and may make assumptions to correct concealed left ankle 922 position according to concealing left knee 920 position. Another option for the system in this situation may be not regarding left ankle 922 at all in its calculations.

Similarly, mid-postures between initial and final postures may be defined. Their parameters may be stored in stored set of values 910 and may be calculated and compared by processor 908. The calculation may be performed on each captured frame of the patient, or less, depending on the exercise nature.

Also for appropriate performance of an exercise, a certain time from initial posture 900 to final posture 902, time for transition between mid-postures, and time for sustaining in final posture 902 may be required. Processor 908 may calculate these time values and compare them to the values stored set of values 910.

### Post Gesture Calculation

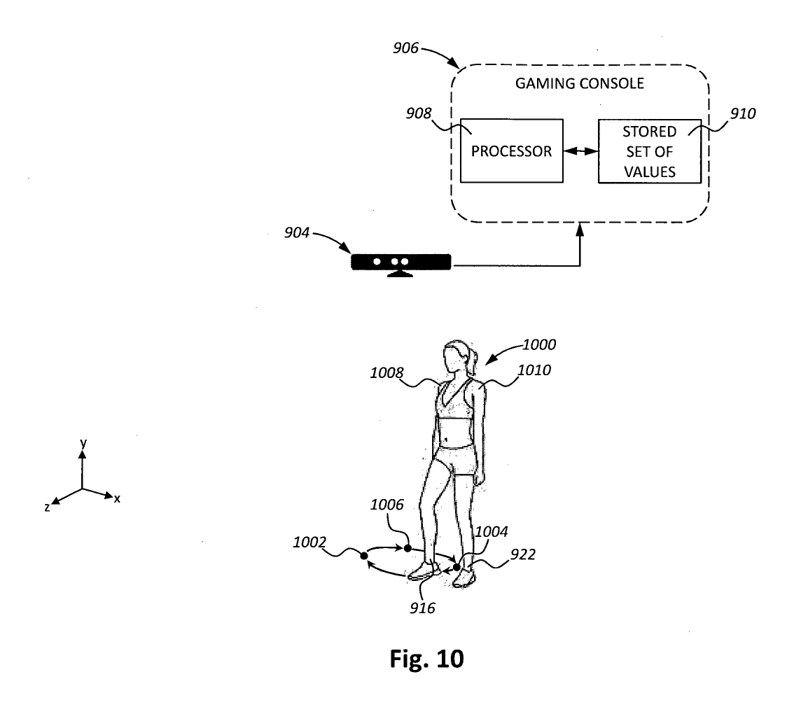

Reference is now made to FIG. 10, which shows an illustration of a right pendulum exercise monitoring. A patient in a right pendulum initial posture 1000 may perform a right pendulum exercise, which may end in the same posture 1000 (i.e. in this exercise the initial and final postures may be identical). In this kind of exercises, post processing may be done by processor 908. In other words, although patient movement may be monitored by motion recognition device 904 and a series of frames may be transferred to gaming console 906 in real time, processor 908 may calculate spatial distances regarding patient movement and compare them to stored set of values 910 only when the final posture of the exercise is identified. In the depicted example, for appropriate performance of a right pendulum, a certain initial posture 1000 may be required. The calculation of initial posture 1000 requirements may be similar to the calculation of initial posture 900, described in a previous example (right lunge exercise). As said before, as final posture may be identical to initial posture 1000, it may have the same requirements. In right pendulum exercise, the patient may be required to perform a circle-like motion with his or her right ankle 916. The imaginary circle may have a high point 1002, in which right ankle 916 is closest to motion recognition device 904 on z axis, a low point 1004, in which right ankle 916 is farthest from motion recognition device 904 on z axis, and a side point 1006, in which right ankle 916 is farthest from patient body on x axis. These points may be required to be on a certain chronological sequence: high point 1002 may be required to appear before side point 1006, which may be required to appear before low point 1004. The distance between high point 1002 and low point 1006 on z axis (also referred as the height of the movement) may be required to be in a certain range. The distance between side point 1006 and the opposite side point on x axis (also referred as the width of the movement) may be required to be in a certain range. The difference between the height and the width may be required to be in a certain range (i.e. the pendulum movement is circle-like enough). Z values of side point 1006 and the opposite side point may be required to be similar, and the difference between this segment and the width of the movement may be required to be within a certain range. Y values of side point 1006 and high point 1002 may be required to have a sufficient difference, similarly to the y values of side point 1006 and the supporting left ankle 922 (i.e. patient right leg did not touch the floor during the exercise). Also for appropriate performance of an exercise, both of patient legs may be required to be straight, and patient shoulders 1008 and 1010 may be required to not lean to the sides.

Also for appropriate performance of an exercise, a certain time from initial posture 1000 to final posture 1000 may be required. Processor 908 may calculate these time values and compare them to the values stored set of values 910.

### Joints Temporal Relation Calculation

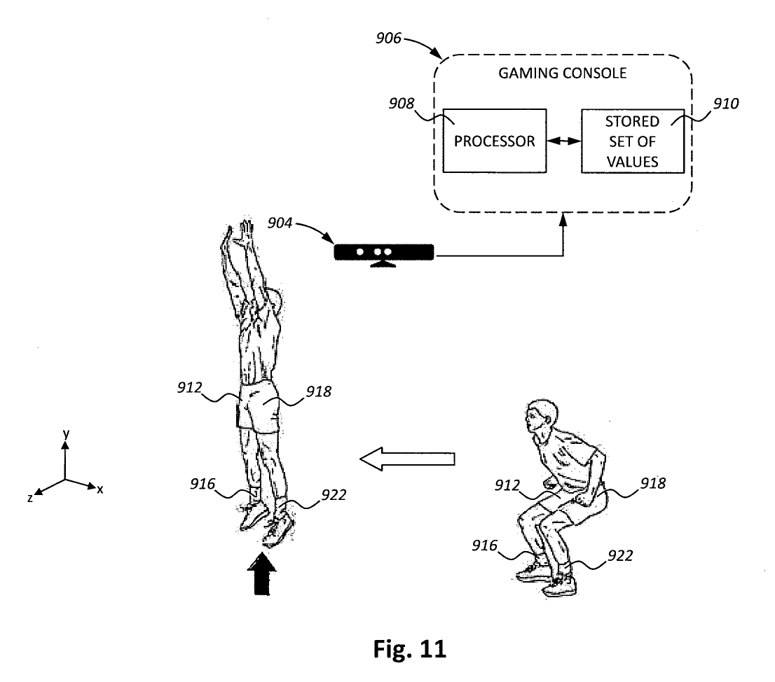

Reference is now made to FIG. 11, which shows an illustration of double leg jump exercise monitoring. In this kind of exercises, the spatial relations between the patient joints may remain similar during the exercise. In other words, there may not be much of a movement of a certain joint in relation to one or more other joints. Thus, in these cases, a reliable way to calculate if the exercise was performed correctly may be to find a spatial relation between a certain joint location and the same joint location at a different time. Namely, to find a difference between a current location of certain joints and their predecessor location. In the double leg jump example, right and left hips (912 and 918) and right and left ankles (916 and 922) may be monitored, since their location may have a significant difference during the exercise, especially on y axis. If an upwards tendency of these joints may be monitored after a satisfying previous posture was achieved, the difference between the y values of these joints and their initial y values may be required to be in a certain range, until exceeding a certain threshold, to determine a jump. When a downwards tendency may be recognized, conditions for final posture may be sought. The double leg jump may end with a final posture, which is actually immediately after landing. Z and y values of right and left ankles (916 and 922) may be required to be similar.

### Combined Calculation

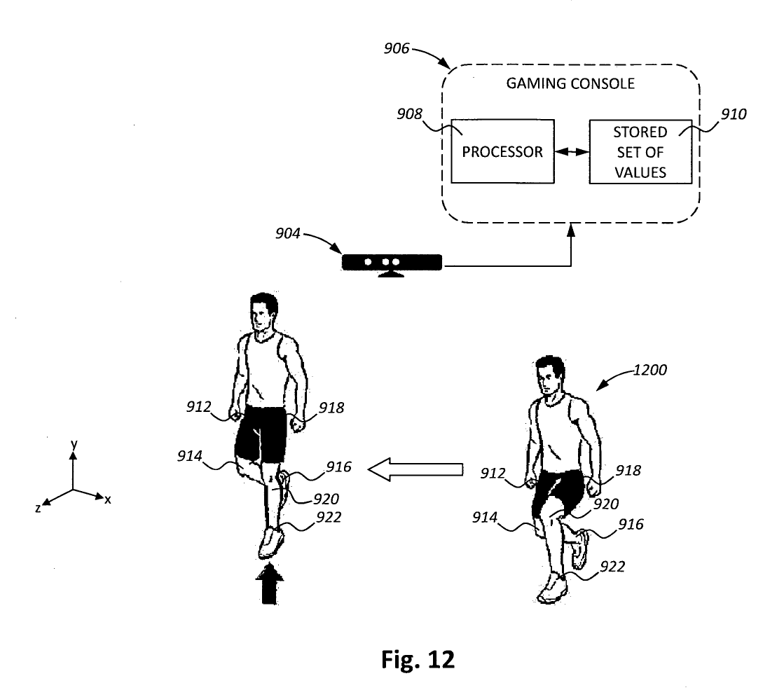

Reference is now made to FIG. 12, which shows an illustration of a left leg jump exercise monitoring. A patient in a left leg jump initial posture 1200 may perform a left leg jump exercise, which may end in the same posture 1200 (i.e. in this exercise the initial and final postures may be identical). Initial (and final) posture 1200 may actually be a left leg stance. As said before, as final posture may be identical to initial posture 1200, they may have the same requirements. In the case of a single (right or left) leg jump, if one or more of the following joints: right and left hips (912 and 918), right and left knees (914 and 920), and right and left ankles (916 and 922) may not be recognized by motion recognition device 904, no other calculations may be done, to avoid false gesture recognition. While performing the jump, the calculation may take into account similar considerations as described in a previous example (double leg jump exercise). In other words, left hip 918 and left ankle 922 may be monitored, since their location may have a significant difference during the exercise, especially on y axis. If an upwards tendency of these joints may be monitored after a satisfying initial posture 1200 was achieved, the difference between the y values of these joints and their initial y values may be required to be in a certain range, until exceeding a certain threshold, to determine a jump. When a downwards tendency may be recognized, conditions for final posture may be sought.

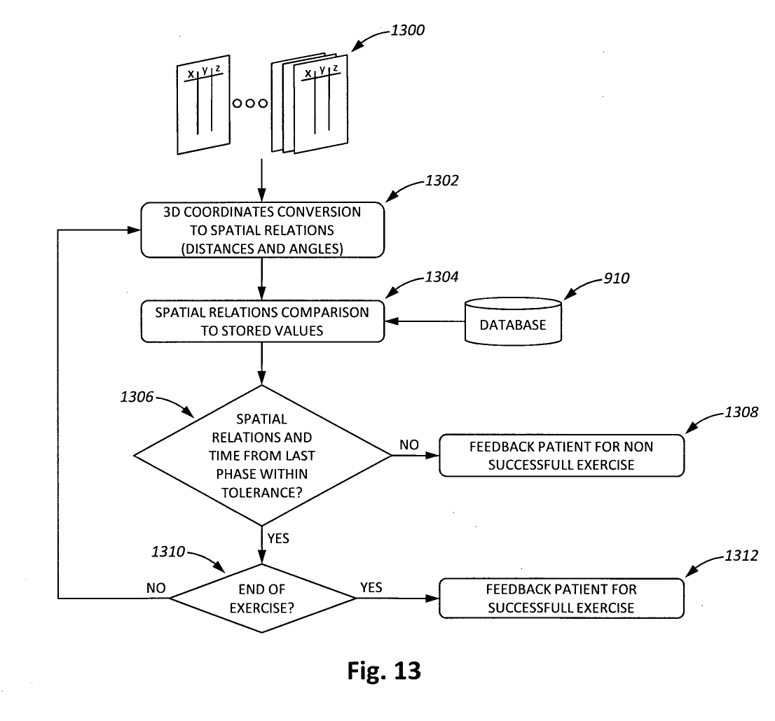

Reference is now made to FIG. 13, which shows a block diagram of gesture detection method. A time series of frames 1300 may be continuously received. Each frame may hold three dimensional position of each of a plurality of patient body joints (i.e. x,y,z coordinates). The coordinates may be then converted 1302 to spatial relations between body limbs and/or joints (i.e. distances between limbs and/or joints, and/or angles between vectors formed by limbs and/or joints) for each captured frame. The spatial relations may be then compared 1304 to corresponding data in database 910. Since a spatial relation may have a range (also stored in database 910), the spatial relations extracted from frames 1300 may vary within their ranges, and still be considered to depict a phase of a successful exercise. Since the way of performing the exercise may be highly important, the order of exercise phases and time between them may have a great significance. Thus, the transition time between each identified exercise phase, which may be checked at each frame or less, may need to be within a range also. If checking ranges 1306 yields a negative result, that phase of the exercise may have not been performed correctly by the patient, and a non success feedback 1308 may be displayed to the patient in a form of a textual and/or graphical message. If checking ranges 1306 yields a positive result, an “end of exercise” check 1310 may be performed, to determine if the last “approved” exercise phase is the last one in the exercise. If yes, the exercise may have ended, and a success feedback 1312 may be displayed to the patient in a form of a textual and/or graphical message. If no, the exercise may have not ended yet, and additional frames may yet have to be converted 1302 to finish the exercise phases sequence.

The present system and method have been described above in connection with a right lunge, pendulum, double leg jump and left leg jump exercises by way of example only. Similarly, the method and system may be used to monitor a variety of other rehabilitative exercises in a similar way.

For a hip flexion exercise, for example, the system may check the execution for the following incorrect performing reasons: side leaning, supporting knee bending, loss of balance (i.e. hand-floor contact), non-adequate hip lifting, exercise short duration, etc.

For a classic squat (on both legs) exercise, for example, the system may check the execution for the following incorrect performing reasons: side leaning, knees turning inwards, asymmetric performance, non-adequate knee bending, loss of balance (i.e. hand-floor contact), exercise short duration, etc.

For a single leg squat exercise, for example, the system may check the execution for the following incorrect performing reasons: side leaning, supporting knee turning inwards, loss of balance (i.e. hand-floor contact), non-adequate knee bending, etc.

For a single leg stance exercise, for example, the system may check the execution for the following incorrect performing reasons: side leaning, supporting knee bending, loss of balance (i.e. hand-floor contact), non-adequate hip lifting, exercise short duration, etc.

In the description and claims of the application, each of the words “comprise” “include” and “have”, and forms thereof, are not necessarily limited to members in a list with which the words may be associated. In addition, where there are inconsistencies between this application and any document incorporated by reference, it is hereby intended that the present application controls.

1. A kinetic rehabilitation system comprising:
a kinetic sensor comprising a motion-sensing camera to detect a patient's gestures; and
a computing device comprising:
(a) a non-transient memory comprising a stored set of values of rehabilitative gestures each defined by a time series of spatial relations between a plurality of theoretical body joints, wherein each rehabilitative gesture comprises gesture phases including at least an initial gesture phase, a mid-gesture phase and a final gesture phase, and wherein each time series of spatial relations for a rehabilitative gesture comprises: initial spatial relations, mid-gesture spatial relations and final spatial relations, and
(b) a hardware processor configured to: (i) automatically translate a therapy plan provided for the patient to a video game level, (ii) continuously receive a recorded time series of frames from said motion-sensing camera during a game play, wherein each frame comprises a three-dimensional position of each of a plurality of body joints of a patient, (iii) compare, in real time, at least a portion of the recorded time series of frames with the time series of spatial relations, to detect an initial gesture phase, a mid-gesture phase and a final gesture phase of a rehabilitative gesture performed by the patient, and (iv) provide real time feedback to the patient during the game play regarding the performed rehabilitative gesture.

2. The system according to claim 1, wherein each of said time series of spatial relations further comprises one or more range values for each of at least one of said spatial relations.

3. The system according to claim 1, wherein said time series of spatial relations further comprises one or more range values for the transition time between each of at least one of said spatial relations.

4. The system according to claim 1, wherein said spatial relations each comprise angles between vectors formed in a three-dimensional space by said theoretical body joints.

5. The system according to claim 1, wherein said spatial relations comprise distances in a three-dimensional space between said theoretical body joints.

6. The system according to claim 1, wherein said motion-sensing camera is configured to yield said recorded time series of frames at a frame rate of 20 frames per second or more.

7. The system according to claim 1, wherein said motion-sensing camera is configured to yield said recorded time series of frames at a frame rate of 30 frames per second or more.

8. The system according to claim 1, wherein said motion-sensing camera is configured to yield said recorded time series of frames at a frame rate of 40 frames per second or more.

9. The system according to claim 1, wherein said hardware processor is further configured to convert said three-dimensional position in said recorded time series of frames to angles between vectors formed in a three-dimensional space by said theoretical body joints.

10. The system according to claim 1, wherein said hardware processor is further configured to convert said three-dimensional position in said recorded time series of frames to distances in a three-dimensional space between said theoretical body joints.

11. A method for gesture detection in a kinetic rehabilitation system, the method comprising:
providing a stored set of values of rehabilitative gestures each defined by time series of spatial relations between a plurality of theoretical body joints, wherein each rehabilitative gesture comprises gesture phases including at least an initial gesture phase, a mid-gesture phase and a final gesture phase, wherein each time series of spatial relations for a rehabilitative gesture comprises: initial spatial relations, mid-gesture spatial relations and final spatial relations; and
using at least one hardware processor for:
(a) automatically translating a therapy plan provided for the patient to a video game level,
(b) continuously receiving a recorded time series of frames from a motion-sensing camera, wherein each frame comprises a three-dimensional position of each of a plurality of body joints of a patient,
(c) comparing, in real time, at least a portion of the recorded time series of frames with the time series of spatial relations, to detect an initial gesture phase, a mid-gesture phase and a final gesture phase of a rehabilitative gesture performed by the patient
(d) providing real time feedback to the patient during the game play regarding the performed rehabilitative gesture.

12. The method according to claim 11, wherein each of said time series of spatial relations further comprises one or more range values for each of at least said spatial relations.

13. The method according to claim 11, wherein each of said time series of spatial relations further comprises one or more range values for the transition time between each of at least said spatial relations.

14. The method according to claim 11, wherein said spatial relations comprise angles between vectors formed in a three-dimensional space by said theoretical body joints.

15. The method according to claim 11, wherein said spatial relations comprise distances in a three-dimensional space between said theoretical body joints.

16. The method according to claim 11, wherein said continuously receiving a recorded time series of frames from a motion-sensing camera is done at a rate of 20 frames per second or more.

17. The method according to claim 11, wherein said continuously receiving a recorded time series of frames from a motion-sensing camera is done at a rate of 30 frames per second or more.

18. The method according to claim 11, wherein said continuously receiving a recorded time series of frames from a motion-sensing camera is done at a rate of 40 frames per second or more.

19. The method according to claim 11, further comprising using the at least one hardware processor for converting said three-dimensional position in said recorded time series of frames to angles between vectors formed in a three-dimensional space by said theoretical body joints.

20. The method according to claim 11, further comprising using the at least one hardware processor for converting said three-dimensional position in said recorded time series of frames to distances in a three-dimensional space between said theoretical body joints.
